(19) United States
(12) Patent Application Publication
(10) Pub. No.: US 2025/0213753 A1
(43) Pub. Date: Jul. 3, 2025

(54) IMPLANT AND METHOD FOR PREPARING THE SAME

(71) Applicant: Industrial Technology Research Institute, Hsinchu (TW)

(72) Inventors: Yu-Bing LIOU, Hsinchu City (TW); Chia-Jung LU, Taichung City (TW); Hsin-Yi HSU, Taoyuan City (TW); Yi-Hsuan LEE, Taipei City (TW); Pei-I TSAI, Hsinchu City (TW)

(73) Assignee: Industrial Technology Research Institute, Hsinchu (TW)

(21) Appl. No.: 18/955,473

(22) Filed: Nov. 21, 2024

Related U.S. Application Data
(60) Provisional application No. 63/615,895, filed on Dec. 29, 2023.

(30) Foreign Application Priority Data
Dec. 29, 2023 (TW) 112151583

(51) Int. Cl. A61L 29/06; A61L 29/08; A61L 29/14

(52) U.S. Cl. CPC A61L 29/06; A61L 29/085; A61L 29/14; A61L 2400/10; A61L 2400/18; A61L 2420/02

(57) ABSTRACT

An implant and a method for preparing the same are provided. The method for preparing the implant includes subjecting a thermoplastic polyurethane to a plasma process to obtain a modified thermoplastic polyurethane. Subsequently, the modified thermoplastic polyurethane is subjected to a water process using a solution to obtain the implant. The solution used in the process is either a hyaluronic acid aqueous solution or a polyvinylpyrrolidone aqueous solution.

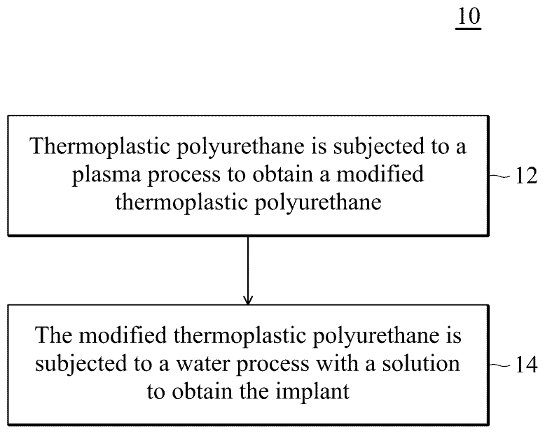

### CROSS REFERENCE TO RELATED APPLICATIONS

This application claims priority of Taiwan Patent Application No. 112151583, filed on Dec. 29, 2023, the entirety of which is incorporated by reference herein. This application claims priority to U.S. Provisional Application Ser. No. 63/615,895, filed on Dec. 29, 2023, the entirety of which is incorporated by reference herein.

### TECHNICAL FIELD

The disclosure relates to an implant and method for preparing the same.

### BACKGROUND

During ureteral stent placement, common complications include postoperative infection, bleeding, pain, etc. The occurrence of such complications can be reduced by surface treatment of the ureteral stent.

One of the ureteral surface treatment methods is surface coating. The method involves forming a hydrophilic monolayer coating on the surface of thermoplastic polyurethane (TPU), which is used as the material for ureteral stents, resulting in reducing the friction between the ureteral stent and the muscle tissue. Although the surface coating method is simple, the hydrophilic coating easily peels off from the thermoplastic polyurethane surface, resulting in poor stability. In addition, a surface treatment of ureteral stent materials is proposed to graft hydrophilic polyelectrolytes onto the thermoplastic polyurethane surface. Although grafting hydrophilic polyelectrolyte onto the thermoplastic polyurethane surface may effectively improve the wettability and stability of the material, the surface grafting method requires the prior chemical grafting of an initiator onto the material surface. This not only complicates the process steps and increases costs but also makes it difficult to remove residual initiators, potentially resulting in toxic process wastewater, thereby limiting the application.

Therefore, there is a need of a novel thermoplastic polyurethane surface treatment to be applied in medical materials for solving the aforementioned problems.

### SUMMARY

According to embodiments of the disclosure, the disclosure provides a method for preparing an implant. The method for preparing an implant includes following steps. First, a thermoplastic polyurethane is subjected to a plasma process to obtain a modified thermoplastic polyurethane. Next, the modified thermoplastic polyurethane is subjected to a water process with a solution to obtain the implant, wherein the solution may be a hyaluronic acid aqueous solution or a polyvinylpyrrolidone aqueous solution.

According to embodiments of the disclosure, the disclosure also provides an implant. The implant is prepared from the method for preparing an implant of the disclosure. The implant includes a hydrophilicity thermoplastic polyurethane, wherein the implant has a water contact angle of less than or equal to 20 degrees, and the implant has a friction coefficient of less than or equal to 0.5.

### BRIEF DESCRIPTION OF THE DRAWINGS

The present disclosure can be more fully understood by reading the subsequent detailed description and examples with references made to the accompanying drawings, wherein:

FIGURE is a flow chart illustrating the method for preparing the implant according to an embodiment of the disclosure.

### DETAILED DESCRIPTION

The implant and method for preparing the same of the disclosure are described in detail in the following description. In the following detailed description, for purposes of explanation, numerous specific details and embodiments are set forth in order to provide a thorough understanding of the present disclosure. The specific elements and configurations described in the following detailed description are set forth in order to clearly describe the present disclosure. It will be apparent, however, that the exemplary embodiments set forth herein are used merely for the purpose of illustration, and the inventive concept may be embodied in various forms without being limited to those exemplary embodiments. In addition, the drawings of different embodiments may use like and/or corresponding numerals to denote like and/or corresponding elements in order to clearly describe the present disclosure. However, the use of like and/or corresponding numerals in the drawings of different embodiments does not suggest any correlation between different embodiments. As used herein, the term “about” in quantitative terms refers to plus or minus an amount that is general and reasonable to persons skilled in the art.

Furthermore, the use of ordinal terms such as “first”, “second”, “third”, etc., in the disclosure to modify an element does not by itself connote any priority, precedence, or der of one claim element over another or the temporal order in which it is formed, but are used merely as labels to distinguish one claim element having a certain name from another element having the same name (but for use of the ordinal term) to distinguish the claim elements.

It should be noted that the elements or devices in the drawings of the disclosure may be present in any form or configuration known to those skilled in the art. In addition, the expression “a layer overlying another layer”, “a layer is disposed above another layer”, “a layer is disposed on another layer”, and “a layer is disposed over another layer” may refer to a layer that directly contacts the other layer, and they may also refer to a layer that does not directly contact the other layer, there being one or more intermediate layers disposed between the layer and the other layer.

The disclosure provides an implant and method for preparing the same.

According to embodiments of the disclosure, by means of the specific plasma process used in concert with the specific water process, a thermoplastic polyurethane with high surface hydrophilicity and low surface friction coefficient can be prepared from the method for preparing an implant of the disclosure. In comparison with conventional technologies, the method for preparing an implant of the disclosure does not involve complex multi-stage chemical reactions, thereby achieving the purpose of simplifying the process. Furthermore, unlike conventional technologies, the method for preparing an implant of the disclosure does not require the use of chemical grafting initiators or crosslinking agents in the process, thereby increasing the stability of the implant (i.e. the implant on day 180 after preparation still has a water contact angle of less than 20 degrees).

According to embodiments of the disclosure, as shown in FIGURE, the method for preparing an implant of the disclosure 10 includes following steps. First, a thermoplastic polyurethane is subjected to a plasma process to obtain a modified thermoplastic polyurethane (step 12). Next, the modified thermoplastic polyurethane is subjected to a water process with a solution to obtain the implant (step 14). According to embodiments of the disclosure, the solution may be a hyaluronic acid aqueous solution or a polyvinylpyrrolidone aqueous solution. According to embodiments of the disclosure, after performing the water process, the obtained implant may optionally be washed (such as washed with water) and dried (such as dried at 40° C.-100° C. to remove water).

According to embodiments of the disclosure, the thermoplastic polyurethane used in the disclosure is not specifically limited and may be a block copolymer obtained by the reaction of conventional isocyanate and dihydric alcohol. According to embodiments of the disclosure, the molecular weight of the thermoplastic polyurethane is not specifically limited. For example, the weight average molecular weight (Mw) of the thermoplastic polyurethane may be about 5,000 g/mol to 3,000,000 g/mol, such as about 8,000 g/mol to 2,500,000 g/mol, 10,000 g/mol to 2,300,000 g/mol, 15,000 g/mol to 2,000,000 g/mol, 10,000 g/mol to 1,000,000 g/mol, 10,000 g/mol to 500,000 g/mol, or 10,000 g/mol to 300,000 g/mol, but the disclosure is not limited thereto. The weight average molecular weight (Mw) of the thermoplastic polyurethane of the disclosure can be determined by gel permeation chromatography (GPC) (using polystyrene as a standard reference to make a calibration curve).

According to embodiments of the disclosure, the plasma process of the disclosure may be performed under a gas, wherein the gas includes argon. According to some embodiments of the disclosure, in order to further increase the stability of the prepared implant, the gas is argon. According to some embodiments of the disclosure, the gas does not contain oxygen.

According to embodiments of the disclosure, the method for preparing an implant of the disclosure utilizes a plasma generator to perform the plasma process, wherein the plasma generator can include a deposition chamber and a radio frequency generator. According to embodiments of the disclosure, the thermoplastic polyurethane material is placed in the deposition chamber of the plasma generator, where the radio frequency generator ionizes the argon gas to generate plasma, and the plasma is used to perform surface treatment on the thermoplastic polyurethane material.

According to embodiments of the disclosure, the radio frequency power of the plasma process may be 300 W to 1,500 W, such as 400 W, 500 W, 600 W, 700 W, 800 W, 900 W, 1,000 W, 1,100 W, 1,200 W, 1,300 W or 1,400 W, but the disclosure is not limited thereto. According to embodiments of the disclosure, the plasma process period may be 150 seconds to 800 seconds, such as 180 seconds, 200 seconds, 240 seconds, 300 seconds, 360 seconds, 500 seconds, 600 seconds or 700 seconds, but the disclosure is not limited thereto. If the RF power is too high or the plasma process period is too long, the process cost would be increased. On the other hand, if the RF power used is too low or the plasma process period is too short, it will not effectively increase the surface hydrophilicity, stability, and uniformity of the implant, and the surface friction coefficient of the implant would be reduced.

According to embodiments of the disclosure, the water process includes immersing the modified thermoplastic polyurethane (i.e., the thermoplastic polyurethane obtained after the plasma process) in the solution. According to embodiments of the disclosure, the water process period (i.e., the time which the modified thermoplastic polyurethane is immersed in the solution) is at least 10 minutes, such as 10 minutes to 60 minutes, but the disclosure is not limited thereto. If the water process period is too short, it will not effectively increase the surface hydrophilicity and stability, and the surface friction coefficient of the implant would be reduced. On the other hand, if the water process period is too long, the efficiency of the disclosed method for preparing the implant would be reduced.

According to embodiments of the disclosure, the solution used in the water process of the disclosure may be a hyaluronic acid aqueous solution or a polyvinylpyrrolidone aqueous solution. According to embodiments of the disclosure, the hyaluronic acid aqueous solution may include hyaluronic acid and water, wherein the weight ratio of hyaluronic acid to water may be 0.2:100 to 10:100, such as 0.3:100, 0.4:100, 0.5:100, 0.8:100, 1:100, 2:100, 3:100, 4:100, 5:100, 6:100, 7:100, 8:100 or 9:100, but the disclosure is not limited thereto. If the content of hyaluronic acid is too low, it will not effectively increase the surface hydrophilicity and stability, and the surface friction coefficient of the implant would be reduced. On the other hand, if the content of hyaluronic acid is too high, it will make the process difficult to operate. According to embodiments of the disclosure, the polyvinylpyrrolidone aqueous solution may consist of polyvinylpyrrolidone and water. According to embodiments of the disclosure, the polyvinylpyrrolidone aqueous solution can include polyvinylpyrrolidone and water, wherein the weight ratio of polyvinylpyrrolidone to water may be 0.2:100 to 10:100, such as 0.3:100, 0.4:100, 0.5:100, 0.8:100, 1:100, 2:100, 3:100, 4:100, 5:100, 6:100, 7:100, 8:100 or 9:100, but the disclosure is not limited thereto. If the content of polyvinylpyrrolidone is too low, it will not effectively increase the surface hydrophilicity and stability, and the surface friction coefficient of the implant would be reduced. On the other hand, if the content of polyvinylpyrrolidone is too high, the process costs would be increased. According to embodiments of the disclosure, the polyvinylpyrrolidone aqueous solution may consist of polyvinylpyrrolidone and water. According to embodiments of the disclosure, the molecular weight of the hyaluronic acid of the disclosure is not limited. For example, the weight average molecular weight (Mw) of the hyaluronic acid may be about 1,500 g/mol to 3,000,000 g/mol, such as about 5,000 g/mol to 2,500,000 g/mol, 8,000 g/mol to 2,500,000 g/mol, 10,000 g/mol to 2,300,000 g/mol, 15,000 g/mol to 2,000,000 g/mol, 10,000 g/mol to 1,000,000 g/mol, 10,000 g/mol to 500,000 g/mol or 10,000 g/mol to 300,000 g/mol, but the disclosure is not limited thereto. According to embodiments of the disclosure, the molecular weight of the polyvinylpyrrolidone of the disclosure is not limited. For example, the weight average molecular weight (Mw) of polyvinylpyrrolidone may be about 2,000 g/mol to 3,000,000 g/mol, such as about 5,000 g/mol to 2,500,000 g/mol, 8,000 g/mol to 2,500,000 g/mol, 10,000 g/mol to 2,300,000 g/mol, 15,000 g/mol to 2,000,000 g/mol, 10,000 g/mol to 1,000,000 g/mol, 10,000 g/mol to 500,000 g/mol or 10,000 g/mol to 300,000 g/mol, but the disclosure is not limited thereto. The weight average molecular weight (Mw) of the hyaluronic acid or polyvinylpyrrolidone of the disclosure can be determined by gel permeation chromatography (GPC) (using polystyrene as a standard reference to make a calibration curve).

According to embodiments of the disclosure, in the water process of the disclosure, apart from the solution used in the disclosure, no other reagents such as grafting initiators or crosslinking agents are used. According to embodiments of the disclosure, apart from the specified hydrophilic polymers (i.e., hyaluronic acid or polyvinylpyrrolidone), the solution of the disclosure does not include any reagents (such as grafting initiators or crosslinking agents) that would react with thermoplastic polyurethane.

According to embodiments of the disclosure, the method for preparing the implant of the disclosure consists of the following steps. First, a thermoplastic polyurethane is subjected to a plasma process to obtain a modified thermoplastic polyurethane. Next, the modified thermoplastic polyurethane is subjected to a water process with a solution to obtain the implant, wherein the solution used in the process is either a hyaluronic acid aqueous solution or a polyvinylpyrrolidone aqueous solution. According to embodiments of the disclosure, the method for preparing the implant of the disclosure involves surface treatment of thermoplastic polyurethane using a specific plasma process and a specific water process, without including any other processes (such as coating processes or any process using reagents or compounds) for surface treatment of the thermoplastic polyurethane.

According to embodiments of the disclosure, the disclosure also provides an implant prepared from the method for preparing the implant of the disclosure. According to embodiments of the disclosure, the water contact angle of the implant may be about less than or equal to 20 degrees, such as about 5 degrees to 20 degrees, 6 degrees to 19 degrees, 7 degrees to 18 degrees, 7 degrees to 17 degrees, or 7 degrees to 16 degrees, but the disclosure is not limited thereto. According to embodiments of the disclosure, after preparation, the implant may be stored in an ambient atmosphere at room temperature. After 180 days of storage, the water contact angle of the implant can still be about less than or equal to 20 degrees, such as about 5 degrees to 20 degrees, 6 degrees to 19 degrees, 7 degrees to 18 degrees, 7 degrees to 17 degrees, or 7 degrees to 16 degrees, but the disclosure is not limited thereto. According to embodiments of the disclosure, the measurement of the water contact angle may be determined by using a high-speed camera to capture images of the water contacting the surface of the sample within 30 seconds for measurement.

According to embodiments of the disclosure, the implant has a friction coefficient (such as dynamic friction coefficient) of about less than or equal to 0.4, such as about 0.1, 0.15, 0.2, 0.25, 0.3, 0.35, or 0.4, but the disclosure is not limited thereto. The friction coefficient may be determined by a method according to ASTM D1894 (with a slider weight of 200±2 g), and calculated based on the formula for friction force (fₖ=μₖN), wherein fₖ is the friction force, N is the perpendicular force, and μₖ is the friction coefficient.

According to embodiments of the disclosure, the term “implant” refers to a medical device that can be positioned in a body location (such as within a body cavity). For example, body locations may include any passage or cavity within a biological body that guides fluids, including but not limited to, bile duct, pancreatic duct, ureter, esophagus, or blood vessels. According to embodiments of the disclosure, the shape of the implant can be designed as needed, for example, it may be tubular. According to embodiments of the disclosure, the implant may be a catheter, a stent, a cannula, or a feeding tube, but the disclosure is not limited thereto.

Below, exemplary embodiments will be described in detail with reference to the accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

### Plasma Process of Thermoplastic Polyurethane

### Preparation Example 1

A medical-grade thermoplastic polyurethane was placed into the deposition chamber of a plasma generator. Next, under a vacuum environment, argon gas was introduced into the deposition chamber (with a flow rate of 100 SCCM) and then ionized to generate a plasma using a radio frequency generator. The thermoplastic polyurethane was treated with the plasma to undergo a surface treatment (with an RF power of 100 W and a process period of 180 seconds), and then Modified thermoplastic polyurethane (1) was obtained.

### Preparation Example 2

Except that the RF power was increased from 100 W to 500 W, Preparation Example 2 was performed in the same manner as in Preparation Example 1 to obtain Modified thermoplastic polyurethane (2).

### Preparation Example 3

Except that the process period was increased from 180 seconds to 600 seconds, Preparation Example 3 was performed in the same manner as in Preparation Example 1 to obtain Modified thermoplastic polyurethane (3).

### Preparation Example 4

Except that the RF power was increased from 100 W to 500 W, Preparation Example 4 was performed in the same manner as in Preparation Example 1 to obtain Modified thermoplastic polyurethane (4).

### Preparation Example 5

Except that the RF power was increased from 100 W to 1,000 W and the process period was increased from 180 seconds to 300 seconds, Preparation Example 5 was performed in the same manner as in Preparation Example 1 to obtain Modified thermoplastic polyurethane (5).

### Preparation Example 6

Except that the RF power was increased from 100 W to 1,000 W and the process period was increased from 180 seconds to 600 seconds, Preparation Example 6 was performed in the same manner as in Preparation Example 1 to obtain Modified thermoplastic polyurethane (6).

### Preparation Example 7

Except that the RF power was increased from 100 W to 900 W and the process period was increased from 180 seconds to 300 seconds, Preparation Example 7 was performed in the same manner as in Preparation Example 1 to obtain Modified thermoplastic polyurethane (7).

### Preparation Example 8

A medical-grade thermoplastic polyurethane was placed into the deposition chamber of a plasma generator. Next, under a vacuum environment, argon gas and oxygen gas were introduced into the deposition chamber (with a flow rate of 100 SCCM and the ratio of argon gas to oxygen gas was 1:1) and then ionized to generate a plasma using a radio frequency generator. The thermoplastic polyurethane was treated with the plasma to undergo a surface treatment (with an RF power of 900 W and a process period of 300 seconds), and then Modified thermoplastic polyurethane (8) was obtained.

### Evaluation of Water Contact Angle

The water contact angles of Modified thermoplastic polyurethane (1)-(8) and thermoplastic polyurethane were measured (repeated three times, and calculated the average), and the results are shown in Table 1. The measurement of the water contact angle was conducted by using a high-speed camera to capture images of water contacting the surface of the sample within 30 seconds.

TABLE 1

| | | | | | average |
|---|---|---|---|---|---|
| | | | | | water |
| | | flow | RF | process | contact |
| | | rate | power | period | angle |
| | gas | (SCCM) | (w) | (s) | (degrees) |
| non-modified | — | — | — | — | 92.77 |
| thermoplastic | | | | | |
| polyurethane | | | | | |
| Modified | argon | 100 | 100 | 180 | 53.61 |
| thermoplastic | gas | | | | |
| polyurethane (1) | | | | | |
| Modified | argon | 100 | 500 | 180 | 39.07 |
| thermoplastic | gas | | | | |
| polyurethane (2) | | | | | |
| Modified | argon | 100 | 500 | 600 | 28.29 |
| thermoplastic | gas | | | | |
| polyurethane (3) | | | | | |
| Modified | argon | 100 | 1,000 | 180 | 36.84 |
| thermoplastic | gas | | | | |
| polyurethane (4) | | | | | |
| Modified | argon | 100 | 1,000 | 300 | 38.49 |
| thermoplastic | gas | | | | |
| polyurethane (5) | | | | | |
| Modified | argon | 100 | 1,000 | 600 | 21.34 |
| thermoplastic | gas | | | | |
| polyurethane (6) | | | | | |
| Modified | argon | 100 | 900 | 300 | 29.87 |
| thermoplastic | gas | | | | |
| polyurethane (7) | | | | | |
| Modified | argon | 100 | 900 | 300 | 67.52 |
| thermoplastic | gas/ | | | | |
| polyurethane (8) | oxygen | | | | |
| | gas | | | | |

### Preparation of Hydrophilicity Thermoplastic Polyurethane

### Example 1

0.5 parts by weight of hyaluronic acid (HA) (commercially available from Kikkoman with a trade number of FCH-200) was dissolved in 100 parts by weight of water to obtain a hyaluronic acid aqueous solution (i.e. the weight ratio of hyaluronic acid to water is 0.5:100). Next, the Modified thermoplastic polyurethane (7) obtained from Preparation Example 7 was immersed in the hyaluronic acid aqueous solution. After 30 minutes, the thermoplastic polyurethane was taken out, washed, and dried to obtain Hydrophilic thermoplastic polyurethane (1).

### Comparative Example 1

0.5 parts by weight of hyaluronic acid (HA) (commercially available from Kikkoman with a trade number of FCH-200) was dissolved in 100 parts by weight of water to obtain a hyaluronic acid aqueous solution (i.e. the weight ratio of hyaluronic acid to water is 0.5:100). Next, the Modified thermoplastic polyurethane (8) obtained from Preparation Example 8 was immersed in the hyaluronic acid aqueous solution. After 30 minutes, the thermoplastic polyurethane was taken out, washed, and dried to obtain Hydrophilic thermoplastic polyurethane (2).

### Comparative Example 2

20 parts by weight of polyethylene glycol (PEG) (commercially available from Sigma-Aldrich with a trade number of P2139) was dissolved in 100 parts by weight of water to obtain a polyethylene glycol aqueous solution (i.e. the weight ratio of polyethylene glycol to water was 20:100). Next, the Modified thermoplastic polyurethane (6) obtained from Preparation Example 6 was immersed in the hyaluronic acid aqueous solution. After 30 minutes, the thermoplastic polyurethane was taken out, washed, and dried to obtain Hydrophilic thermoplastic polyurethane (3).

### Example 2

10 parts by weight of polyvinylpyrrolidone (PVP) (commercially available from Sigma-Aldrich with a trade number of P2472) was dissolved in 100 parts by weight of water to obtain a polyvinylpyrrolidone aqueous solution (i.e. the weight ratio of polyvinylpyrrolidone to water was 10:100). Next, the Modified thermoplastic polyurethane (6) obtained from Preparation Example 6 was immersed in the hyaluronic acid aqueous solution. After 30 minutes, the thermoplastic polyurethane was taken out, washed, and dried to obtain Hydrophilic thermoplastic polyurethane (4).

### Example 3

0.25 parts by weight of hyaluronic acid (HA) (commercially available from Kikkoman with a trade number of FCH-200) was dissolved in 100 parts by weight of water to obtain a hyaluronic acid aqueous solution (i.e. the weight ratio of hyaluronic acid to water was 0.25:100). Next, the Modified thermoplastic polyurethane (6) obtained from Preparation Example 6 was immersed in the hyaluronic acid aqueous solution. After 30 minutes, the thermoplastic polyurethane was taken out, washed, and dried to obtain Hydrophilic thermoplastic polyurethane (5).

### Evaluation of Water Contact Angle

The water contact angles of Hydrophilicity thermoplastic polyurethane (1)-(5) were measured (repeated three times, and calculated the average), and the results are shown in Table 2.

TABLE 2

| | | | average |
|---|---|---|---|
| | | | water |
| | | | contact |
| | | | angle |
| | material | water process | (degrees) |
| Hydrophilicity | Modified | hyaluronic acid aqueous solution | 14.59 |
| thermoplastic | thermoplastic | (hyaluronic acid:water = 0.5:100) | |
| polyurethane (1) | polyurethane (7) | | |
| Hydrophilicity | Modified | hyaluronic acid aqueous solution | 68.69 |
| thermoplastic | thermoplastic | (hyaluronic acid:water = 0.5:100) | |
| polyurethane (2) | polyurethane (8) | | |
| Hydrophilicity | Modified | polyethylene glycol (PEG) | 30.41 |
| thermoplastic | thermoplastic | aqueous solution | |
| polyurethane (3) | polyurethane (6) | (polyethylene glycol | |
| | | (PEG):water = 20:100) | |
| Hydrophilicity | Modified | polyvinylpyrrolidone aqueous | 15.95 |
| thermoplastic | thermoplastic | solution | |
| polyurethane (4) | polyurethane (6) | (polyvinylpyrrolidone:water = | |
| | | 10:100) | |
| Hydrophilicity | Modified | hyaluronic acid aqueous solution | 9.82 |
| thermoplastic | thermoplastic | (hyaluronic acid:water = 0.25:100) | |
| polyurethane (5) | polyurethane (6) | | |

### Evaluation of Friction Coefficient

The friction coefficient of Hydrophilicity thermoplastic polyurethane (3)-(5) and thermoplastic polyurethane were measured (repeated three times, and calculated the average), and the results are shown in Table 3. The friction coefficient was determined by a method according to ASTM D1894, and calculated based on the formula for friction force (fₖ=μₖN), where fₖ is the friction force, N is the perpendicular force, and μₖ is the friction coefficient.

TABLE 3

| | | | friction |
|---|---|---|---|
| | material | water process | coefficient |
| non-modified | — | — | 0.73 |
| thermoplastic | | | |
| polyurethane | | | |
| Hydrophilicity | Modified | polyethylene glycol | 0.44 |
| thermoplastic | thermoplastic | (PEG) aqueous solution | |
| polyurethane | polyurethane | (polyethylene glycol | |
| (3) | (6) | (PEG):water = 20:100) | |
| Hydrophilicity | Modified | polyvinylpyrrolidone | 0.21 |
| thermoplastic | thermoplastic | aqueous solution | |
| polyurethane | polyurethane | (polyvinylpyrrol- | |
| (4) | (6) | idone:water = 10:100) | |
| Hydrophilicity | Modified | hyaluronic acid | 0.31 |
| thermoplastic | thermoplastic | aqueous solution | |
| polyurethane | polyurethane | (hyaluronic | |
| (5) | (6) | acid:water = 0.25:100) | |

As shown in Table 1, although the water contact angle of thermoplastic polyurethane surface could be reduced via the plasma treatment, the effect is limited. Namely, the water contact angle of thermoplastic polyurethane, which is merely subjected to a plasma process, could not be reduced to be less than 20 degrees. As shown in Table 2, after subjecting the thermoplastic polyurethane to an argon gas plasma process and subsequently subjecting the modified thermoplastic polyurethane to a water process with a hyaluronic acid aqueous solution or a polyvinylpyrrolidone aqueous solution, the water contact angle of the obtained hydrophilic thermoplastic polyurethane could be further reduced to be less than 20 degrees. In addition, as shown in Table 3, the hydrophilic thermoplastic polyurethane prepared from the method of the disclosure employing the argon gas plasma process and water process (with specific aqueous solutions) exhibits a significantly reduced surface friction coefficient.

### Example 4

5 parts by weight of polyvinylpyrrolidone (PVP) (commercially available from Sigma-Aldrich with a trade number of P2474) was dissolved in 100 parts by weight of water to obtain polyvinylpyrrolidone aqueous solution (i.e. the weight ratio of polyvinylpyrrolidone to water was 5:100). Next, the Modified thermoplastic polyurethane (6) obtained from Preparation Example 6 was immersed in the hyaluronic acid aqueous solution. After 30 minutes, the thermoplastic polyurethane was taken out, washed, and dried to obtain Hydrophilic thermoplastic polyurethane (6).

### Evaluation of Stability

The water contact angle of Hydrophilicity thermoplastic polyurethane (6) was measured (repeated three times, and calculated the average), and the results are shown in Table 4. Next, the hydrophilic thermoplastic polyurethane (6) was stored in the ambient atmosphere at room temperature. During the storage, the water contact angle of Hydrophilicity thermoplastic polyurethane (6) was measured on day 7, day 14, day 30, day 90, and day 180 (repeating the measurement three times and calculating the average), and the results are shown in Table 4.

TABLE 4

| time (days) | water contact angle (degrees) |
|---|---|
| 0 | 16.98 |
| 7 | 11.07 |
| 14 | 11.83 |
| 30 | 11.79 |
| 90 | 17.15 |
| 180 | 7.59 |

As shown in Table 4, the hydrophilic thermoplastic polyurethane prepared from the method of the disclosure employing the argon gas plasma process and water process (with specific aqueous solutions) exhibits a high surface stability of at least 6 months (i.e., the water contact angle of the hydrophilic thermoplastic polyurethane of the disclosure is less than 20 degrees after being stored for 6 months).

Accordingly, by means of the specific plasma process used in concert with the specific water process, a thermoplastic polyurethane with high surface hydrophilicity and low surface friction coefficient can be prepared from the method for preparing an implant of the disclosure. In comparison with conventional technologies, the method for preparing an implant of the disclosure does not involve complex multi-stage chemical reactions, thereby achieving the purpose of simplifying the process.

While the disclosure has been described by way of example and in terms of the preferred embodiments, it should be understood that the disclosure is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for preparing an implant, comprising:
subjecting a thermoplastic polyurethane to a plasma process to obtain a modified thermoplastic polyurethane; and
subjecting the modified thermoplastic polyurethane to a water process with a solution to obtain the implant, wherein the solution used in the water process is either a hyaluronic acid aqueous solution or a polyvinylpyrrolidone aqueous solution.

2. The method for preparing an implant as claimed in claim 1, wherein the implant has a water contact angle of less than or equal to 20 degrees.

3. The method for preparing an implant as claimed in claim 1, wherein the implant on day 180 after preparation has a water contact angle of less than or equal to 20 degrees.

4. The method for preparing an implant as claimed in claim 1, wherein the implant has a friction coefficient of less than or equal to 0.4.

5. The method for preparing an implant as claimed in claim 1, wherein the plasma process is performed under a gas, and wherein the gas comprises argon.

6. The method for preparing an implant as claimed in claim 1, wherein the plasma process has a radio frequency power of 300 W to 1,500 W.

7. The method for preparing an implant as claimed in claim 1, wherein the plasma process has a process period of 150 seconds to 800 seconds.

8. The method for preparing an implant as claimed in claim 1, wherein the water process has a process period of 10 minutes to 60 minutes.

9. The method for preparing an implant as claimed in claim 1, wherein the hyaluronic acid aqueous solution comprises hyaluronic acid and water, and the weight ratio of hyaluronic acid to water is 0.2:100 to 10:100.

10. The method for preparing an implant as claimed in claim 1, wherein the polyvinylpyrrolidone aqueous solution comprises polyvinylpyrrolidone and water, and the weight ratio of polyvinylpyrrolidone to water is 0.2:100 to 10:100.

11. An implant, which is prepared by the method as claimed in claim 1, comprising:
a hydrophilicity thermoplastic polyurethane, wherein the implant has a water contact angle of less than or equal to 20 degrees, and the implant has a friction coefficient of less than or equal to 0.5.

12. The implant as claimed in claim 11, wherein the implant on day 180 after preparation has a water contact angle of less than or equal to 20 degrees.

13. The implant as claimed in claim 11, wherein the implant is tubular.

14. The implant as claimed in claim 11, wherein the implant is a catheter, a stent, a cannula or a feeding tube.
